(19) United States
(12) Patent Application Publication
(10) Pub. No.: US 2025/0281185 A1
(43) Pub. Date: Sep. 11, 2025

(54) VASCULAR CLIP FOR BLOOD FLOW CONTROL

(71) Applicants: KOREA UNIVERSITY RESEARCH AND BUSINESS FOUNDATION, Seoul (KR); SEOUL NATIONAL UNIVERSITY HOSPITAL, Seoul (KR)

(72) Inventors: Jahyun KOO, Seoul (KR); Won-Sang CHO, Seoul (KR); Sungeun KIM, Seoul (KR)

(73) Assignees: KOREA UNIVERSITY RESEARCH AND BUSINESS FOUNDATION, Seoul (KR); SEOUL NATIONAL UNIVERSITY HOSPITAL, Seoul (KR)

(21) Appl. No.: 18/851,305

(22) PCT Filed: Dec. 21, 2022
(86) PCT No.: PCT/KR2022/020938

§ 371 Date: May 28, 2025

(30) Foreign Application Priority Data
Mar. 28, 2022 (KR) 10-2022-0038036

(51) Int. Cl. A61B 17/122; A61B 17/00; A61L 31/14

(52) U.S. Cl. CPC A61B 17/122; A61L 31/146; A61B 2017/00867; A61B 2017/00955; A61L 2400/16

(57) ABSTRACT

The present invention relates to a vascular clip for blood flow control, wherein the vascular clip is based on a shape-memory polymer and serves to partially occlude a vessel through which excessive blood flow passes due to the incomplete automatic regulatory function thereof so as to induce normal blood flow and release the occlusion when the automatic regulatory function recovers, the occlusion is released, whereby when applied as a device to substitute for the lost automatic regulatory function of the vessel, the vascular clip can prevent cerebral hyperperfusion syndrome in advance, thus solving problems caused by the syndrome and can eliminate the risks of removal surgery, thus providing safety and convenience for patients.

### TECHNICAL FIELD

The present invention relates to a vascular clip for controlling blood flow and, more specifically, to a surgical clip that restores normal blood flow by compressing an in vivo blood vessel and releasing the compression based on a shape memory polymer.

### BACKGROUND ART

Cerebrovascular diseases, which rank first and second in terms of mortality rate in Korea as single diseases, are a group of diseases that are managed under a special law as they cause significant health, social, and economic problems. As the number of patients with cerebrovascular diseases continues to increase due to aging, the problems caused thereby are expected to worsen. For ischemic cerebrovascular disease, which accounts for approximately 80% of cerebrovascular diseases, drugs are primarily used for prevention and treatment, and in cases where the disease is not controlled with the drugs, it is treated through surgery or endovascular procedures. For severe carotid artery or cerebrovascular stenosis, methods such as carotid endarterectomy, stent insertion, or cerebrovascular anastomosis are used for treatment. Since cerebral blood vessels originally have an autoregulatory function, they can maintain cerebral blood flow at a constant level by adjusting the diameter of the blood vessels by themselves even when the supply of blood flow to the brain is insufficient or excessive. However, if chronic blood flow deficiency continues due to ischemic cerebrovascular disease, the cerebral blood vessels become maximally dilated in order to maximize cerebral blood flow and the autoregulatory function is impaired. In this chronic cerebral ischemic state, the dilated cerebral blood vessels that have temporarily lost autoregulatory function do not manage the restored (increased) blood flow immediately after the surgical treatments. Therefore, hyperperfusion (excessively increased cerebral blood flow) occurs. After restoring the autoregulatory function over 1 to 2 weeks, cerebral blood flow can be regulated appropriately. Here, symptoms such as headache, paralysis, and cerebral hemorrhage can appear due to an overload of brain cells caused by hyperperfusion during the period of 1 to 2 weeks immediately after revascularization treatments. This case is called hyperperfusion syndrome.

Various complications, including cerebral hyperperfusion syndrome, may occur after surgery or endovascular procedures are performed on patients with chronic ischemic cerebrovascular disease. The fundamental problem is that there is still no way to assess the status of cerebrovascular autoregulation. Therefore, cerebral hyperperfusion syndrome can occur regardless of day or night from immediately after surgery (procedure) for about two weeks, and thus it can be differentially diagnosed through continuous monitoring by medical staff and various tests. If cerebral hyperperfusion syndrome is not treated promptly, it can develop into a fatal hemorrhagic stroke with a mortality rate of up to 40%.

The most commonly used method to prevent and treat cerebral hyperperfusion syndrome includes controlling blood pressure through medication. In this case, blood pressure is controlled to a level similar to or slightly lower than before treatment (procedure) through medication. However, if blood pressure is controlled to an excessively low level, cerebral infarction may be induced, and if blood pressure is controlled to an excessively high level, cerebral hyperperfusion syndrome may occur. Thus, continuous blood pressure monitoring and the resulting drug administration and blood pressure control are necessary. This leads to increased medical costs, increased fatigue of medical staff, and increased pain and anxiety in patients.

When endovascular stent insertion for carotid artery stenosis is performed, in patients with a high likelihood of developing cerebral hyperperfusion syndrome, staged angioplasty is sometimes performed, in which the blood vessel is not dilated significantly from the beginning, but only half of the blood vessel is dilated and then completely dilated after about 1 to 2 weeks. Although this method has a good preventive effect, it has the disadvantage of being applicable only to some patients who have undergone stenting and not to patients who have undergone surgery (vascular anastomosis, or carotid endarterectomy).

Therefore, there is an urgent need for the development of a medical device that can effectively prevent cerebral hyperperfusion syndrome.

### DISCLOSURE

### Technical Problem

The present invention has been made to solve the above-described problems in the prior art. One aspect of the present invention is to provide a vascular clip for controlling blood flow based on a shape memory polymer. This clip restores normal blood flow by partially ligating a blood vessel where excessive blood flow occurs due to impaired cerebral autoregulation and releasing the ligation when the autoregulatory function is restored.

### Technical Solution

A vascular clip for controlling blood flow, according to an embodiment of the present invention, includes a deformable central portion, a first pressing portion connected to one end of the deformable central portion, and a second pressing portion connected to the other end of the deformable central portion to face the first pressing portion with a blood vessel interposed therebetween. The deformable central portion is formed of a first shape memory polymer that presses the first and second pressing portions in a direction close to each other by shape recovery at body temperature to compress the blood vessel.

In addition, in the vascular clip for controlling blood flow, according to an embodiment of the present invention, each of the first and second pressing portions may include a main body and a core. The main body is connected to the deformable central portion and configured to contact and compress the blood vessel; a core is disposed inside the main body and made of a second shape memory polymer. The cores of the first and second pressing portions cause the main bodies to spread apart from one another due to shape recovery at a reaction temperature exceeding body temperature, thereby relieving the compression of the blood vessel.

In addition, according to an embodiment of the present invention, the vascular clip for controlling blood flow may include a heat source disposed in each of the first and second pressing portions. Heat source is configured to heat the core to the reaction temperature in response to an external stimulus applied from outside the body.

In addition, in the vascular clip for controlling blood flow, according to an embodiment of the present invention, the external stimulus may include at least one of a magnetic field, an electromagnetic wave, and a radio frequency.

In addition, in the vascular clip for controlling blood flow, according to an embodiment of the present invention, the heat source may be composed of either nanoparticles dispersed inside the core or a polymer coating the outer surface of the core. The nanoparticle can generate heat in response to the magnetic field or electromagnetic wave, and the polymer can generate heat in response to electromagnetic waves.

In addition, in the vascular clip for controlling blood flow, according to an embodiment of the present invention, the heat source may be a micro-heater placed on the outer surface of the core and configured to generate heat by the radio frequency.

In addition, in the vascular clip for controlling blood flow according to an embodiment of the present invention, the first and second pressing portions may be formed of a third shape memory polymer to recover their shape in a direction away from each other in response to the body temperature so as to release the compression of the blood vessel having a lower shape recovery rate than that of the deformable central portion.

In addition, in the vascular clip for controlling blood flow according to an embodiment of the present invention, each of the first and second pressing portions may include a support connected to the deformable central portion and a degradable layer disposed on the outer surface of the support and configured to contact and compress the blood vessel. The degradable layer may biodegrade after a predetermined period of time to release the compression of the blood vessel.

In addition, in the vascular clip for controlling blood flow according to an embodiment of the present invention, the support may biodegrade slower than the degradable layer.

In addition, in the vascular clip for controlling blood flow according to an embodiment of the present invention, the degradable layer may have a porous or mesh structure.

In addition, in the vascular clip for controlling blood flow according to an embodiment of the present invention, the deformable central portion may have a deformation-promoting groove recessed inward from a space between the first pressing portion and the second pressing portion.

In addition, in the vascular clip for controlling blood flow according to an embodiment of the present invention, each of the first pressing portion and the second pressing portion may have a blood vessel-contact surface formed to be convex toward the blood vessel.

The features and advantages of the present invention will become more apparent from the following detailed description taken with reference to the accompanying drawings.

The terms or words used in the specifications and claims should not be construed as usual or dictionary definition but should be rather construed to be consistent with the technical spirits of the present invention based on the principle that the inventor may properly define the terms to describe his/her invention in the best manner.

### Advantageous Effects

The vascular clip for controlling blood flow according to the present invention is made of a shape memory polymer material, and thus it is convenient to use as it is placed in contact with a blood vessel by itself within a few minutes in response to body temperature. In addition, the vascular clip remains in contact with the blood vessel while compressing the blood vessel for 1 to 2 weeks, and after the expected (functional) life thereof is over, the structure thereof degrades by itself and detaches from the blood vessel. Accordingly, the vascular clip may be applied as a medical device that replaces the lost autoregulatory function of blood vessels, which is the primary cause of cerebral hyperperfusion syndrome, thereby preventing cerebral hyperperfusion syndrome in advance and thus solving problems caused by cerebral hyperperfusion syndrome.

In addition, the structure of the vascular clip may degrade non-invasively and the vascular clip is made of a biodegradable shape memory polymer. Thus, according to the present invention, it is possible to provide safety and convenience to patients by eliminating the risk of removal surgery.

### BRIEF DESCRIPTION OF DRAWINGS

FIGS. 1 to 3 illustrate a vascular clip for controlling blood flow and the operating process thereof according to a first embodiment of the present invention.

FIG. 4 illustrates a vascular clip for controlling blood flow and the operating process thereof according to a second embodiment of the present invention.

FIGS. 5 and 6 schematically illustrate a vascular clip for controlling blood flow according to a third embodiment of the present invention.

FIG. 7 illustrates a vascular clip for controlling blood flow and the operating process thereof according to a fourth embodiment of the present invention.

FIG. 8 illustrates a vascular clip for controlling blood flow and the operating process thereof according to a fifth embodiment of the present invention.

FIG. 9 depicts photographs showing an experimental method according to an experimental example.

FIG. 10 depicts photographs showing the results of the experimental example.

### BEST MODE

The objects, specific advantages, and novel features of the present invention will become more apparent from the following detailed description and preferred embodiments taken in conjunction with the accompanying drawings. It should be noted that, when adding reference numerals to components in each drawing in the present invention, identical components are given the same reference numerals as much as possible even if they are shown in different drawings. In addition, terms such as “first,” “second,” etc. are used to distinguish one component from another component, and the components are not limited by these terms. In the following description of the present invention, a detailed description of related known technologies will be omitted if it may unnecessarily obscure the subject matter of the present invention.

Hereinafter, preferred embodiments of the present invention will be described in detail with reference to the accompanying drawings.

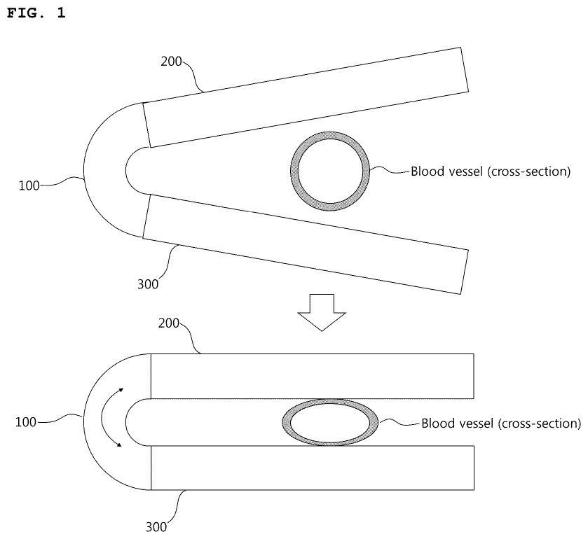

FIGS. 1 to 3 illustrate a vascular clip for controlling blood flow and the operating process thereof according to a first embodiment of the present invention.

As illustrated in FIGS. 1 to 3, the vascular clip for controlling blood flow according to the first embodiment of the present invention includes: a deformable central portion 100; a first pressing portion 200 connected to one end of the deformable central portion 100; and a second pressing portion 300 connected to the other end of the deformable central portion 100 so as to face the first pressing portion 200 with a blood vessel interposed therebetween, wherein the deformable central portion 100 is formed of a first shape memory polymer that presses the first pressing portion 200 and the second pressing portion 300 in a direction close to each other by shape recovery at body temperature so as to compress the blood vessel.

The present invention is a vascular clip for controlling blood flow, which may be used to partially and temporarily (for example, for 1 to 2 weeks) ligate a donor blood vessel for the purpose of preventing cerebral hyperperfusion syndrome that commonly occurs after cerebrovascular anastomosis or carotid endarterectomy performed to treat ischemic cerebrovascular disease. However, the present invention is not necessarily used only for the purpose of preventing cerebral hyperperfusion syndrome, but may also be widely used in cases where blood vessels need to be compressed to control blood flow within the blood vessels.

As used herein, “ligation” does not mean completely tying off a blood vessel to stop blood flow, but means compressing the blood vessel to reduce the cross-sectional area perpendicular to the direction of blood flow within the blood vessel (the same applies hereinafter).

Specifically, the vascular clip for controlling blood flow according to the first embodiment of the present invention includes a deformable central portion 100, a first pressing portion 200, and a second pressing portion 300.

The deformable central portion 100 is a member that supports the first pressing portion 200 and the second pressing portion 300, in which one end thereof is connected to the first pressing portion 200 and the other end is connected to the second pressing portion 300. Here, the deformable central portion 100 supports the first pressing portion 200 and the second pressing portion 300 so that the first pressing portion 200 and the second pressing portion 300 face each other at a predetermined angle.

The deformable central portion 100 is formed of a first shape memory polymer. The shape memory polymer has the property of recovering its shape from a temporarily deformed secondary shape to its original primary shape in response to stimuli such as temperature (heat), light, electromagnetic waves, ultrasound, and pH. The first shape memory polymer forming the deformable central portion 100 is a material that recovers its original shape in response to a body temperature of about 35° C. to 40° C. Although the deformable central portion 100 formed of the first shape memory polymer is in a state in which it is deformed so that the angle between the first pressing portion 200 and the second pressing portion 300 widens to the extent that a blood vessel may be inserted between the first pressing portion 200 and the second pressing portion 300, it is originally formed so that the first pressing portion 200 and the second pressing portion 300 maintain an angle therebetween that is sufficient to compress a blood vessel. Thus, when the deformable central portion 100 responds to body temperature, it recovers its original shape and, at the same time, presses the first pressing portion 200 and the second pressing portion 300 in a direction close to each other so as to compress the blood vessel. That is, the deformable central portion 100 is in a state in which it is deformed so that the curvature of the curve connecting one end thereof and the other end decreases, and then it recovers its original shape in response to body temperature so that the curvature increases.

Through this shape recovery of the deformable central portion 100, the vascular clip for controlling blood flow according to the present invention may ligate a blood vessel without any external force.

Here, the shape recovery of the deformable central portion 100 at body temperature is achieved within several minutes, and the first shape memory polymer having such shape recovery ability may include at least one selected from the group consisting of NOA63, PGS-UPy, poly(tert-butyl acrylate), PMM, MP3510, MDI-PBA-BDO, IPDI-PCL-BDO, POSS-PDLLA, and MM3520. However, the first shape memory polymer is not necessarily limited thereto, and any temperature-responsive shape memory polymer that recovers its original shape at body temperature may be used without particular limitation.

The first pressing portion 200 and the second pressing portion 300 are members that compress the blood vessel placed therebetween while moving against each other by shape recovery of the deformable central portion 100.

Each of the first pressing portion 200 and the second pressing portion 300 has a contact surface that contacts a blood vessel, and the shape of the contact surface may be formed to be convex toward the blood vessel or to be flat, depending on the thickness of the blood vessel wall.

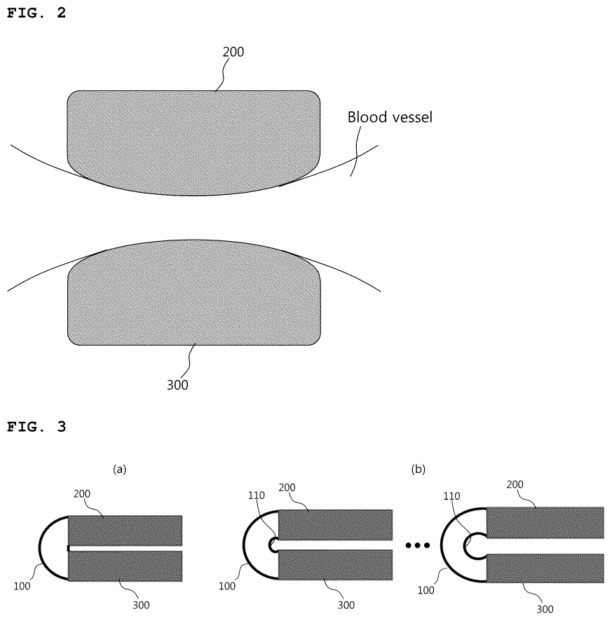

Referring to FIG. 2, the convex contact surface is suitable in the case where the target blood vessel has a thin blood vessel wall. That is, the convex contact surface may be used in the case where the target blood vessel has a thin blood vessel wall and thus the concentration of stress caused by clip ligation and the resulting generation of turbulence may cause blood vessel damage. When the vascular clip having the convex contact surface is selected and applied, the cross-sectional area of the blood vessel at the blood vessel-contact surface of the clip used for ligation may be gradually decreased and then gradually increased along the blood flow direction, and stress may be evenly distributed over a large area of the blood vessel wall, thereby minimizing the load applied to the blood vessel wall.

The flat contact surface is suitable for a blood vessel with a thick blood vessel wall. Due to the thick blood vessel wall of the target blood vessel, the vascular clip needs to have a strong compressive force in order to reduce blood flow to a desired extent by ligating the blood vessel by the vascular clip, so it is preferable to use a vascular clip with a flat contact surface.

In conclusion, since the thickness of the blood vessel wall is different for each type of blood vessel and each patient, a clip having a blood vessel-contacting surface of a different shape depending on the thickness of the blood vessel wall of each target blood vessel may be selected and used to stably and effectively ligate the blood vessel.

Referring to FIG. 3, the deformable central portion 100 may have a deformation-promoting groove 110 depending on the diameter of the blood vessel. The deformation-promoting groove 110 is formed to be recessed from the space between the first pressing portion 200 and the second pressing portion 300 into the deformable central portion 100. Since the diameter of a blood vessel is different for each type of blood vessel and each patient, a vascular clip having the deformation-promoting groove 100 of a different size depending on the diameter of each target blood vessel or not having the deformation-promoting groove 100 may be selected and applied.

The vascular clip having the deformation-promoting groove 110 in the deformable central portion 100 is suitable in the case where a target blood vessel with a large diameter is to be ligated. When placing the clip around a blood vessel, the first pressing portion 200 and the second pressing portion 300 should be spread apart wide. At this time, deformation occurs while the deformable central portion 100 is stretched (curvature is reduced), and the deformation is facilitated by forming the deformation-promoting groove 110. In addition, ligation of the blood vessel with the clip should be performed to only partially reduce the cross-sectional area of the blood vessel rather than completely blocking the blood vessel, and thus when the deformation-promoting groove 110 is formed, it may provide a sufficient space between the first pressing portion 200 and the second pressing portion 300 of the clip used for ligation, thereby reducing blood flow to a desired level.

The formation of the deformation-promoting groove 110 in the deformable central portion 100 is not essential in all cases. When a target blood vessel with a small diameter is to be ligated, a clip without the deformation-promoting groove 110 may be used. When placing the clip around a blood vessel with a small diameter, the first pressing portion 200 and the second pressing portion 300 of the clip may be spread slightly, and thus the clip may be easily deformed even if the deformation-promoting groove 110 is not formed in the deformable central portion 100. In addition, if there is the deformation-promoting groove 110 in the deformable central portion 100, when the clip is used for ligation, the space between the first pressing portion 200 and the second pressing portion 300 of the clip may be too large, so that a sufficient compressive force may not be provided to the blood vessel and a desired degree of blood flow reduction may not be achieved. Therefore, when a target blood vessel with a diameter of 1 mm or less is to be ligated, it is preferable to select and apply a clip without the deformation-promoting groove 110 in the deformable central portion 100.

Meanwhile, the vascular clip for controlling blood flow according to the present invention may release blood vessel compression (ligation) by itself. Hereinafter, the present invention will be described focusing on an example in which blood vessel compression is released.

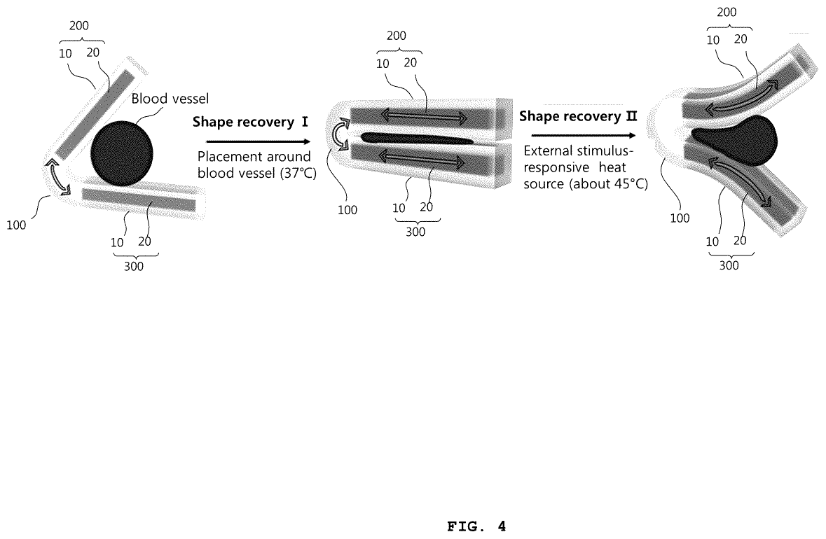

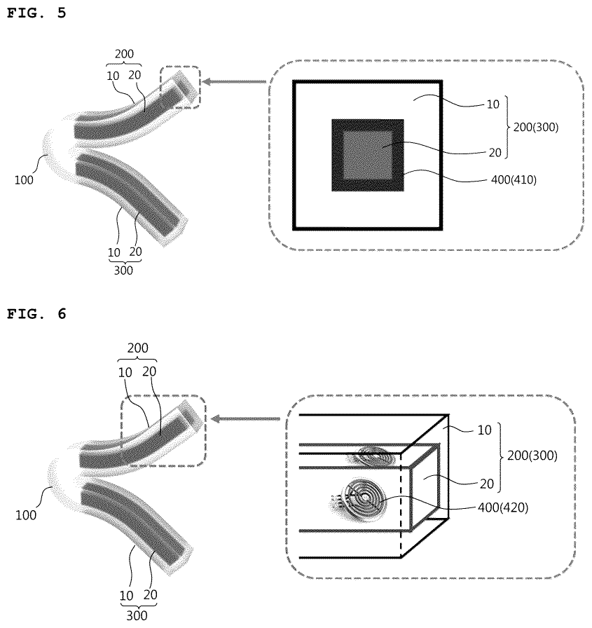

FIG. 4 illustrates a vascular clip for controlling blood flow and the operating process thereof according to a second embodiment of the present invention. Referring thereto, each of the first pressing portion 200 and the second pressing portion 300 of the vascular clip for controlling blood flow according to the second embodiment of the present invention may include a main body 10 and a core 20.

The main body 10 is a member connected to the deformable central portion 100 and configured to directly contact and compress a blood vessel. This main body 10 is a flexible member that is bendable in response to a change in the shape of the core 20 disposed therein. The main body 10 may be formed of the same first shape memory polymer as that of the deformable central portion 100. However, since the main body 10 has the same primary shape and secondary shape, it does not change its shape in response to body temperature.

The core 20 is a member having a predetermined length and is disposed inside the main body 10 along the longitudinal direction of the main body 10. The core 20 is formed of a second shape memory polymer that responds to temperature changes and recovers its original shape at a temperature higher than body temperature. Here, the core 20 disposed inside the main body 10 of the first pressing unit 200 and the core 20 disposed inside the main body 10 of the second pressing unit 300 recover their shape so as to spread apart from each other. Therefore, the shape-recovering cores 20 of the first pressing portion 200 and the second pressing portion 300 cause the main body 10 of the first pressing portion 200 and the main body 10 of the second pressing portion 300 to spread apart from each other, and thus the gap between the first pressing portion 200 and the second pressing portion 300 is widened, thereby releasing the compression of the blood vessel. The temperature at which the shape recovery of the cores 20 occurs is preferably about 45° C., which is higher than the shape recovery temperature of the deformable central portion 100. However, the temperature at which the shape recovery of the cores 20 occurs is not necessarily limited thereto and may be any temperature at which the shape recovery of the cores may be distinguished from the shape recovery of the deformable central portion 100. The second shape memory polymer may include at least one selected from the group consisting of poly-L-lactic acid (PLA), polyurethanes (PUs), and poly(ε-caprolactone) (PCL).

The first shape memory polymer and the second shape memory polymer may be biodegradable polymers. Among the first and second shape memory polymers exemplified above, PGS-UPy, polyurethane (PU), poly-L-lactic acid (PLA), polyesters, poly(ε-caprolactone) (PCL), etc., are biodegradable polymers. If a bio-implanted medical device remains permanently in the body, a problem may arise in that the medical device migrates and damages surrounding tissues, or the immune response to the foreign substance (permanent implant) is excessively activated, resulting in foreign body reaction, chronic inflammation, etc. For this reason, a second surgery is performed to remove the permanent implant, but there are risks such as surgically-induced infection, difficulty in removal, and damage to surrounding tissues. In particular, surgery to remove a medical device implanted around a cerebral blood vessel can cause fatal side effects that can cause secondary damage to surrounding brain tissues. In contrast, when a vascular clip is formed of a biodegradable shape memory polymer according to the present invention, it may be degraded after performing its function and absorbed in the body or discharged, thereby eliminating the risk of removal surgery and providing safety and convenience to the patient.

Meanwhile, for shape recovery of the cores 20, a heat source 400 may be employed as a means for heating the cores 20 to a reaction temperature required for the shape recovery.

FIGS. 5 and 6 schematically illustrate a vascular clip for controlling blood flow according to a third embodiment of the present invention, and the heat source 400 will be described with reference thereto.

The heat source 400 is disposed in the first pressing portion 200 and the second pressing portion 300, and can heat the cores 20 to a reaction temperature at which the cores 20 can recover their original shape in response to an external stimulus applied from outside the body. Here, the external stimulus may include at least one of a magnetic field, an electromagnetic wave, and a radio frequency.

For example, the heat source 400 may be nanoparticles dispersed inside the cores 20. These nanoparticles may be magnetic nanoparticles such as iron oxide nanoparticles. If magnetic nanoparticles are dispersed inside the cores 20, when a magnetic field is applied from outside the body, the magnetic nanoparticles may vibrate and generate heat, so that the temperature of the cores 20 may be increased and their original shape may be recovered, thereby releasing ligation by the clip. However, the nanoparticles are not necessarily limited to magnetic nanoparticles, and may be nanoparticles that generate heat in response to electromagnetic waves such as near-infrared rays applied from outside the body. Such nanoparticles are nanoparticles such as gold (Au), graphene, and graphene oxide nanoparticles, and absorb light energy through the photothermal effect and release the same as heat energy.

As another example, as shown in FIG. 5, the heat source 400 may be provided using a polymer that generates heat when exposed to electromagnetic waves. Specifically, the heat source 400 may be provided by coating the outer surface of each core 20 with a polymer exhibiting a photothermal effect to form a polymer layer 410.

As another example, as shown in FIG. 6, the heat source 400 may be a micro-heater 420. This micro-heater 420 is disposed on the outer surface of each core 20 and may be operated by a radio frequency such as RF applied from the outside, thereby supplying heat to each core 20.

Meanwhile, where the cores 20 are heated by the heat source 400, each main body 10 acts as an insulating layer to prevent thermal damage to living tissue, because each main body 10 surrounds each core 20.

According to the present invention, release of the compression of the blood vessel is possible without using the heat source 400 activated by the external stimulus described above. This will be described below.

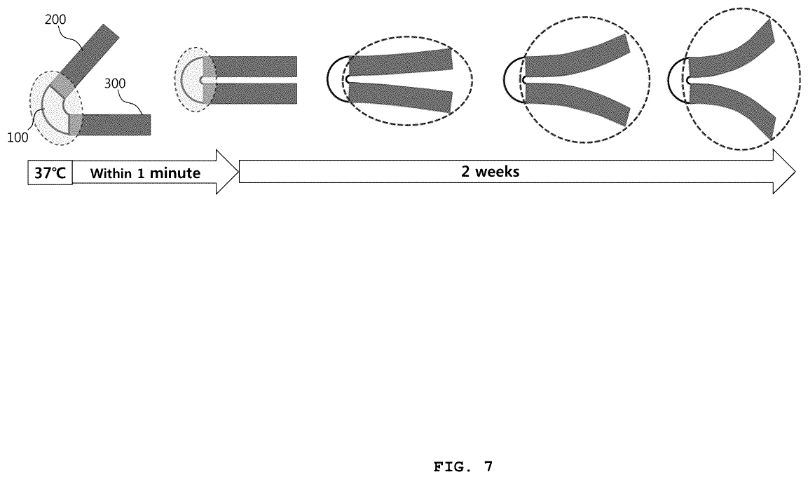

FIG. 7 illustrates a vascular clip for controlling blood flow and the operating process thereof according to a fourth embodiment of the present invention. As illustrated in FIG. 7, each of the first pressing portion 200 and the second pressing portion 300 of the vascular clip for controlling blood flow according to the fourth embodiment of the present invention may be formed of a third shape memory polymer. Here, the third shape memory polymer is a thermoresponsive shape memory polymer that recovers its original shape at body temperature. The first pressing portion 200 and the second pressing portion 300, which are formed of the third shape memory polymer may recover their original shape at body temperature so as to spread apart from each other, thereby releasing the compression of the blood vessel. However, the shape recovery rate of these portions is lower than that of the deformable central portion 100. For example, the deformable central portion 100 may recover its original shape within a few minutes in the body, thereby ligating the blood vessel, and the first pressing portion 200 and the second pressing portion 300 may recover their original shape gradually after several days, thereby releasing the ligation of the blood vessel. This third shape memory polymer may be selected from among shape memory polymers that recover their shape in the temperature range of 40° C. to 60° C., and may be tailored to slowly recover its shape over a period of about two weeks by controlling conditions such as crosslinking density, type of crosslinking agent, monomer chain length and concentration, and deformation temperature. Examples of the third shape memory polymer include poly(tert-buty acrylate) with 10 wt % or less of DEGDMA, poly(ε-caprolactone), polyurethane, MDI-PEA-BDO, IPDI-PCL-BDO, PEU, MM4510, MM4520, MM5520 (SMP Technologies), Veriflex styrene (Cornerstone Research Group), etc.

In conclusion, according to the fourth embodiment of the present invention, release of the ligation of the blood vessel by the clip is induced without the above-described heat source 400 (see FIGS. 5 and 6) by controlling the shape recovery rates of the first shape memory polymer and the third shape memory polymer. By synthesizing a shape memory polymer to have an increased content of a linear monomer having a linear chain structure, it is possible to reduce the shape recovery rate of the shape memory polymer. Conversely, by using a different type of crosslinker to increase the glass transition temperature, or by adjusting the content, etc., to reduce the crosslinking density, or by increasing the deformation temperature, it is possible to increase the shape recovery time, thereby controlling the shape recovery rates of the first shape memory polymer and the third shape memory polymer.

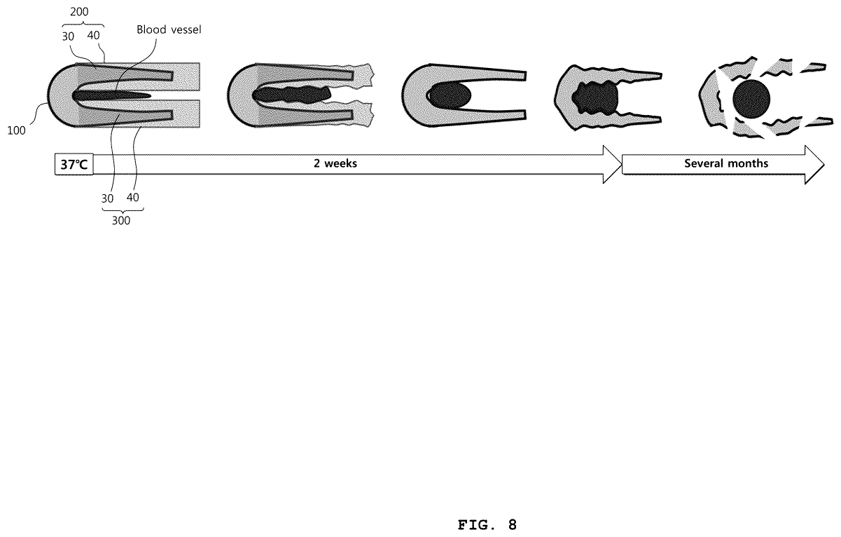

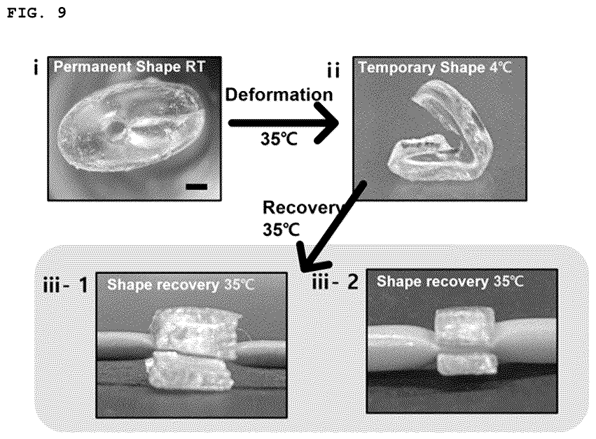

FIG. 8 illustrates a vascular clip for controlling blood flow and the operating process thereof according to a fifth embodiment of the present invention. Referring to FIG. 8, the blood flow control vascular clip according to the fifth embodiment of the present invention makes it possible to release the ligation of the blood vessel by the clip without the heat source 400 (see FIGS. 5 and 6) by using a biodegradable material.

Specifically, each of the first pressing portion 200 and the second pressing portion 300 of the vascular clip for controlling blood flow according to the fifth embodiment of the present invention may include a support 30 and a degradable layer 40. Here, the support 30 is a member connected to the deformable central portion 100. For example, the support 30 may be formed of the same material as that of the deformable central portion 100 and may extend from each of one end and the other end of the deformable central portion 100. The degradable layer 40 is disposed on the outer surface of the support 30 and configured to directly contact and compress the blood vessel. This degradable layer 40 is formed of a biodegradable material and may biodegrade after a certain period of time, thereby releasing the compression of the blood vessel.

In addition, the support 30 may also be formed of a biodegradable material, and in this case, the support 30 may biodegrade slower than the degradable layer 40. In this case, as the degradable layer 40 biodegrades, compression of the blood vessel may be primarily released, and as the support 30 biodegrades, compression of the blood vessel may be completely released. However, it is not necessary that compression of the blood vessel be completely released through degradation of the support 30. In other words, compression of the blood vessel may be completely released through degradation of the degradable layer 40 and then the clip may disappear due to degradation of the support 30.

Biodegradable materials have different biodegradation rates, and the period of time during which the materials can retain their structure before their degradation into fragments (biodegradation life) varies from several days to several months. In addition, it is possible to control the biodegradation rate and life of a biodegradable material by making the biodegradable material have a porous structure or a mesh structure or have different thicknesses in portions thereof (a thin material degrades quickly and disappears).

Meanwhile, where the support 30 and the deformable central portion 100 are made of non-biodegradable polymers, the process in which ligation by clip is released proceeds up to the third step (from the left) shown in FIG. 8. At this time, as the gap between the first pressing portion 200 and the second pressing portion 300 of the clip widens, the blood vessel can come out of the clip. Examples of such non-biodegradable polymers include NOA 63, PMM, poly(tert-butyl acrylate), etc.

Where the support 30 and the deformable central portion 100 are made of biodegradable polymers, the process in which ligation by the clip is released proceeds to the fifth step (from the left) shown in FIG. 8. Examples of such biodegradable polymers include PGS-UPy, MP3510, and MP 3520.

In the case of the degradable layer 40 that biodegrades faster than the support 30, even if a material with a low biodegradation rate itself is used, the biodegradation rate of the material may be increased by making the material have a porous structure, mesh structure, etc. For example, the degradable layer 40 may be formed of a biodegradable polymer having shape memory properties, such as PU including MP3510, PGS-UPy, or the like, or may be formed of a biodegradable polymer without shape memory properties, such as polyanhydrides, including PLGA, PGS, PLLA, PGA, poly(vinyl alcohol) (PVA), and PBTPA. Where the support 30 and the deformable central portion 100 are made of biodegradable polymers, the material of the degradable layer 40 may also be of the same type, but may have a mesh structure, a porous structure, or the like, so that it may biodegrade rapidly. In this case, the first pressing portion 200 and the second pressing portion 300 may be first degraded to become thinner within a few weeks, thereby releasing the ligation of the blood vessel.

### DESCRIPTION OF REFERENCE NUMERALS

10: main body, 20: core, 30: support, 40: degradable layer, 100: deformable central portion, 110: deformation-promoting groove, 200: first pressing portion, 300: second pressing portion, 400: heat source, 410: polymer layer, 420: micro-heater.

### Mode for Invention

Hereinafter, the present invention will be described in more detail by way of an experimental example.

### 1. Experimental Method

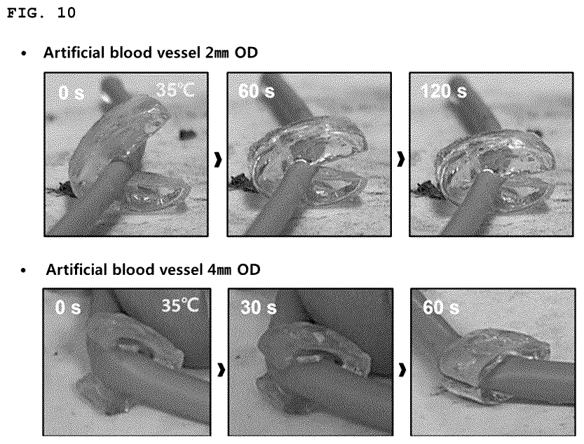

FIG. 9 depicts photographs showing an experimental method according to an experimental example. Referring to FIG. 9, NOA 63 prepolymer was poured into a PDMS mold, and cured by irradiation with UV light (365 nm wavelength) for 1 hour, thereby producing a vascular clip in which the first pressing portion and second pressing portion connected to one end and the other end of the deformable central portion, respectively, are disposed close to each other. Next, the deformable central portion of the clip was deformed by stretching while applying a heat of 35° C. so that the curvature thereof was reduced. To fix this temporary deformation, the clip was placed in a refrigerator at 4° C. An experiment was conducted, in which the deformed clip with the first and second pressing portions spreading apart from each other was placed back at a temperature of 35° C., and the clip recovered to its permanent shape, thereby ligating the artificial blood vessel by the clip. Here, the photograph on the left shows a state in which a small-sized vascular clip ligates an artificial blood vessel with a diameter of 2 mm, and the photograph on the right shows a state in which a relatively large-sized vascular clip ligates an artificial blood vessel with a diameter of 4 mm. Through this experiment, the present inventors evaluated the convenience of placement and mounting around the blood vessels and the effectiveness of ligation, due to the rapid shape recovery of the material of the deformable central portion at a temperature of 35° C.

### 2. Experimental Results

FIG. 10 depicts photographs showing the results of the experimental example. Referring to FIG. 10, the clip with the fixed temporary deformation (a state in which the first pressing portion and the second pressing portion spread apart from each other) was placed on a hot plate at 35° C., an artificial blood vessel was placed between the first pressing portion and the second pressing portion of the clip, and the shape recovery ability of the clip was observed. As a result, it was confirmed that the small-sized clip for ligating an artificial blood vessel with a diameter of 2 mm ligated the blood vessel within 2 minutes at 35° C., and the relatively large-sized clip for ligating an artificial blood vessel with a diameter of 4 mm ligated the blood vessel within 1 minute at 35° C., suggesting that the clips have the ability to rapidly recover their shape.

Although the present invention has been described in detail with reference to the specific embodiments, these embodiments are intended to describe the present invention in detail, and the present invention is not limited thereto. It will be apparent to those skilled in the art that modifications and improvements are possible without departing from the technical spirit of the present invention.

All simple modifications or changes of the present invention fall within the scope of the present invention, and the specific protection scope of the present invention will become clear from the appended claims.

### Industrial Applicability

The vascular clip for controlling blood flow according to the present invention is made of a shape memory polymer material, and thus it is convenient to use as it is placed in contact with a blood vessel by itself within a few minutes in response to body temperature. In addition, the vascular clip remains in contact with the blood vessel while compressing the blood vessel for 1 to 2 weeks, and after the expected (functional) life thereof is over, the structure thereof degrades by itself and detaches from the blood vessel. Accordingly, the vascular clip may be applied as a medical device that replaces the lost autoregulatory function of blood vessels, and thus is industrially applicable.

1. A vascular clip for controlling blood flow, comprising:
a deformable central portion;
a first pressing portion connected to one end of the deformable central portion; and
a second pressing portion connected to the other end of the deformable central portion so as to face the first pressing portion with a blood vessel interposed therebetween,
wherein the deformable central portion is formed of a first shape memory polymer that presses the first pressing portion and the second pressing portion in a direction close to each other by shape recovery at body temperature so as to compress the blood vessel.

2. The vascular clip of claim 1, wherein each of the first pressing portion and the second pressing portion comprises: a main body connected to the deformable central portion and configured to contact and compress the blood vessel; and
a core disposed inside the main body and formed of a second shape memory polymer, wherein the cores of the first pressing portion and the second pressing portion cause the main bodies to spread apart from each other by shape recovery at a reaction temperature higher than the body temperature so as to release the compression of the blood vessel.

3. The vascular clip of claim 2, further comprising a heat source disposed in each of the first pressing portion and the second pressing portion and configured to heat the core to the reaction temperature in response to an external stimulus applied from outside the body.

4. The vascular clip of claim 3, wherein the external stimulus comprises at least one of a magnetic field, an electromagnetic wave, and a radio frequency.

5. The vascular clip of claim 4, wherein the heat source is composed of either nanoparticles dispersed inside the core and configured to generate heat by the magnetic field or the electromagnetic wave, or a polymer with which an outer surface of the core is coated and which is configured to generate heat by the electromagnetic wave.

6. The vascular clip of claim 4, wherein the heat source is a micro-heater placed on an outer surface of the core and configured to generate heat by the radio frequency.

7. The vascular clip of claim 1, wherein the first pressing portion and the second pressing portion are each formed of a third shape memory polymer, recover their shape in a direction away from each other in response to the body temperature so as to release the compression of the blood vessel, and have a lower shape recovery rate than that of the deformable central portion.

8. The vascular clip of claim 1, wherein each of the first pressing portion and the second pressing portion comprises:
a support connected to the deformable central portion; and
a degradable layer disposed on an outer surface of the support and configured to contact and compress the blood vessel, wherein the degradable layer biodegrades after a predetermined period of time to release the compression of the blood vessel.

9. The vascular clip of claim 8, wherein the support biodegrades slower than the degradable layer.

10. The vascular clip of claim 9, wherein the degradable layer has a porous or mesh structure.

11. The vascular clip of claim 1, wherein the deformable central portion has a deformation-promoting groove recessed inward from a space between the first pressing portion and the second pressing portion.

12. The vascular clip of claim 1, wherein each of the first pressing portion and the second pressing portion has a blood vessel-contact surface formed to be convex toward the blood vessel.
